(19) United States
(12) Patent Application Publication
(10) Pub. No.: US 2014/0276140 A1
(43) Pub. Date: Sep. 18, 2014

(54) Systems and Methods for Multi-Modality Medical Data Collection

(71) Applicant: Volcano Corporation, San Diego, CA (US)

(72) Inventor: Curtis Kinghorn, Oceanside, CA (US)

(73) Assignee: Volcano Corporation, San Diego, CA (US)

(21) Appl. No.: 14/209,818

(22) Filed: Mar. 13, 2014

Related U.S. Application Data
(60) Provisional application No. 61/784,715, filed on Mar. 14, 2013.

(51) Int. Cl. A61B 5/0215; A61B 5/0472; A61B 5/024

(52) U.S. Cl. CPC A61B 5/0215; A61B 5/02405; A61B 5/0472
USPC 600/486; 600/508; 600/521

(57) ABSTRACT

Embodiments of the present disclosure are configured to collect multi-modality medical data from a patient. In some embodiments, a method includes acquiring heartbeat data from the patient using a heart-monitoring device, the heartbeat data identifying a first cardiac cycle of the patient and selecting a diagnostic window within the first cardiac cycle of the patient, wherein the diagnostic window encompasses only a portion of the first cardiac cycle of the patient. The method also includes acquiring first medical data from the patient during the diagnostic window using a first medical device, the first medical data being associated with a first medical modality and acquiring second medical data from the patient during the diagnostic window using a second medical device, the second medical data being associated with a second medical modality different than the first medical modality.

### CROSS-REFERENCE TO RELATED APPLICATIONS

The present application claims priority to and the benefit of U.S. Provisional Patent Application No. 61/784,715, filed Mar. 14, 2013, which is hereby incorporated by reference in its entirety.

### TECHNICAL FIELD

Embodiments of the present disclosure relate generally to the field of medical devices and, more particularly, to multi-modality medical data collection and associated methods and systems. Aspects of the present disclosure are particularly suited for evaluation of biological vessels in some instances.

### BACKGROUND

Innovations in diagnosing and verifying the level of success of treatment of disease have migrated from external imaging processes to internal diagnostic processes. In particular, diagnostic equipment and processes have been developed for diagnosing vasculature blockages and other vasculature disease by means of ultra-miniature sensors placed upon the distal end of a flexible elongate member such as a catheter, or a guide wire used for catheterization procedures. For example, known medical sensing techniques include angiography, intravascular ultrasound (IVUS), forward looking IVUS (FL-IVUS), fractional flow reserve (FFR) determination, a coronary flow reserve (CFR) determination, optical coherence tomography (OCT), trans-esophageal echocardiography, and image-guided therapy. Each of these techniques may be better suited for different diagnostic situations. To increase the chance of successful treatment, health care facilities may have a multitude of imaging, treatment, diagnostic, and sensing modalities on hand in a catheter lab during a procedure.

However, synchronization of data collection during a multi-modality procedure may be difficult as portions of a patient's body may be continually moving during data collection. For example, blood vessels are continuously expanding and relaxing in response to blood being pumped therethrough. In one traditional technique, a peak of a cardiac cycle as captured by an electrocardiogram (ECG) is identified as a trigger for data collection. However, in multi-modality data collection procedures it may not be feasible for every medical instrument to collect data at an instantaneous point. Further, in multi-modality procedures designed to collect data in small vessels such as coronary arteries, dramatic fluctuation in resistance within the vessel may prevent accurate data collection. Although pharmacological hyperemic agents, such as adenosine, may be administered to reduce and stabilize the resistance within the coronary arteries, the administration of such hyperemic agents is not always possible or advisable.

Accordingly, while existing multi-modality medical data acquisition techniques and systems have been generally adequate for their intended purposes, they have not been entirely satisfactory in all respects.

### SUMMARY

The present disclosure is directed to systems and methods for synchronizing the collection of different types of medical data using a portion of a patient's heartbeat cycle. Such synchronization may include identifying a window within a patient's heartbeat cycle, and then collecting multiple types of medical data from the patient during the window.

In one exemplary aspect, the present disclosure is directed to a method for collecting multi-modality medical data from a patient. The method includes acquiring heartbeat data from the patient using a heart-monitoring device, the heartbeat data identifying a first cardiac cycle of the patient and selecting a diagnostic window within the first cardiac cycle of the patient, wherein the diagnostic window encompasses only a portion of the first cardiac cycle of the patient. The method also includes acquiring first medical data from the patient during the diagnostic window using a first medical device, the first medical data being associated with a first medical modality and acquiring second medical data from the patient during the diagnostic window using a second medical device, the second medical data being associated with a second medical modality different than the first medical modality.

In some instances, the method further includes introducing at least one instrument into a vessel of the patient and obtaining from the at least one instrument pressure measurements within the vessel, the pressure measurements collectively defining a waveform of the cardiac cycle of the patient.

In another exemplary aspect, the present disclosure is directed to another a method of collecting multi-modality medical data from a patient. The method includes introducing at least one instrument into a vessel of the patient, obtaining from the at least one instrument pressure measurements within the vessel for at least one cardiac cycle of the patient, and selecting a diagnostic window within the at least one cardiac cycle of the patient, wherein the diagnostic window encompasses only a portion of the at least one cardiac cycle of the patient. The method also includes acquiring first medical data from the patient during the diagnostic window using a first medical device, the first medical data being associated with a first medical modality, acquiring second medical data from the patient during the diagnostic window using a second medical device, the second medical data being associated with a second medical modality different than the first medical modality, and co-registering the first medical data with the second medical data.

In another exemplary aspect, the present disclosure is directed to a multi-modality medical data collection system. The system includes a heart-monitoring device, a first medical device configured to acquire first medical data associated with a first medical modality, a second medical device configured to acquire second medical data associated with a second medical modality different than the first medical modality, and a multi-modality processing system in communication with the heart-monitoring device, the first medical device, and the second medical device. The processing system is configured to acquire heartbeat data from a patient using the heart-monitoring device, the heartbeat data identifying a first cardiac cycle of the patient and select a diagnostic window within the first cardiac cycle of the patient, wherein the diagnostic window encompasses only a portion of the first cardiac cycle of the patient. The processing system is also configured to control the first medical device to acquire the first medical data from the patient during the diagnostic window and control the second medical device to acquire the second medical data from the patient during the diagnostic window.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic drawing depicting a medical system including a multi-modality processing system according to one embodiment of the present disclosure.

FIG. 2 is a diagrammatic perspective view of a vessel having a stenosis according to an embodiment of the present disclosure.

FIG. 3 is a diagrammatic, partial cross-sectional perspective view of the vessel of FIG. 2 with instruments positioned therein according to an embodiment of the present disclosure.

FIG. 4 is a diagrammatic, schematic view of a system according to an embodiment of the present disclosure.

FIG. 5 is a graphical representation of measured pressure, velocity, and resistance within a vessel according to an embodiment of the present disclosure.

FIG. 6 is a magnified view of a portion of the graphical representation of FIG. 5 corresponding to a resting state of a patient.

FIG. 7 is a magnified view of a portion of the graphical representation of FIG. 5 corresponding to a hyperemic state of a patient.

FIG. 8 is the portion of the graphical representation of FIG. 6 annotated to identify a diagnostic window according to an embodiment of the present disclosure.

FIG. 9 is a graphical representation of measured pressure and velocity within a vessel according to an embodiment of the present disclosure.

FIG. 10 is a graphical representation of a derivative of the measured velocity of FIG. 9 according to an embodiment of the present disclosure.

FIG. 11 is the graphical representation of FIG. 9 annotated to identify a diagnostic window according to an embodiment of the present disclosure.

FIG. 12 is a graphical representation of wave intensity within a vessel according to an embodiment of the present disclosure.

FIG. 13 is a graphical representation of proximal and distal originating pressure waves within a vessel corresponding to the wave intensity of FIG. 12 according to an embodiment of the present disclosure.

FIG. 14 is a graphical representation of pressure and velocity within a vessel corresponding to the wave intensity of FIG. 12 and the proximal and distal originating pressure waves of FIG. 13 according to an embodiment of the present disclosure.

FIG. 15 is a graphical representation of a resistance within a vessel corresponding to the wave intensity of FIG. 12, the proximal and distal originating pressure waves of FIG. 13, and the pressure and velocity of FIG. 14 according to an embodiment of the present disclosure.

FIG. 16 is a graphical representation of an identification of a starting point of a diagnostic window based on a proximal pressure measurement according to an embodiment of the present disclosure.

FIG. 17 is a graphical representation of an identification of a starting point of a diagnostic window based on a proximal pressure measurement according to another embodiment of the present disclosure.

FIG. 18 is a graphical representation of an identification of a starting point of a diagnostic window based on a proximal pressure measurement according to another embodiment of the present disclosure.

FIG. 19 is a graphical representation of an identification of a starting point of a diagnostic window based on a distal pressure measurement according to an embodiment of the present disclosure.

FIG. 20 is a graphical representation of an identification of a starting point of a diagnostic window based on a distal pressure measurement according to another embodiment of the present disclosure.

FIG. 21 is a graphical representation of an identification of a starting point of a diagnostic window based on a distal pressure measurement according to another embodiment of the present disclosure.

FIG. 22 is a graphical representation of an identification of a starting point of a diagnostic window based on a distal pressure measurement according to another embodiment of the present disclosure.

FIG. 23 is a graphical representation of an identification of an ending point of a diagnostic window based on a starting point of the diagnostic window according to an embodiment of the present disclosure.

FIG. 24 is a graphical representation of an identification of an ending point of a diagnostic window based on a proximal pressure measurement according to an embodiment of the present disclosure.

FIG. 25 is a graphical representation of an identification of an ending point of a diagnostic window based on a distal pressure measurement according to an embodiment of the present disclosure.

FIG. 26 is a graphical representation of an identification of an ending point of a diagnostic window based on a distal pressure measurement according to an embodiment of the present disclosure.

FIG. 27 is a graphical representation of a diagnostic window relative to proximal and distal pressure measurements according to an embodiment of the present disclosure.

FIG. 28 is a graphical representation of a diagnostic window relative to proximal and distal pressure measurements according to another embodiment of the present disclosure.

FIG. 29 is graphical representation of an ECG signal according to an embodiment of the present disclosure.

FIG. 30 is a graphical representation of a diagnostic window relative to proximal and distal pressure measurements according to another embodiment of the present disclosure.

FIG. 31 is a graphical representation of a procedure timeline associated with a multi-modality data collection procedure according to an embodiment of the present disclosure.

FIG. 32 is a graphical representation of another procedure timeline associated with a multi-modality data collection procedure

FIG. 33 is a graphical representation of yet another procedure timeline associated with a multi-modality data collection procedure

FIG. 34 is a simplified flow chart describing a method for collecting multi-modality data from a patient according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

FIG. 1 is a schematic drawing depicting a medical system 100 including a multi-modality processing system 101 according to one embodiment of the present disclosure. In general, the medical system 100 provides for coherent integration and consolidation of multiple forms of acquisition and processing elements designed to be sensitive to a variety of methods used to acquire and interpret human biological physiology and morphological information. More specifically, in system 100, the multi-modality processing system 101 is an integrated device for the acquisition, control, interpretation, and display of multi-modality medical data. In one embodiment, the processing system 101 is a computer system with the hardware and software to acquire, process, and display multi-modality medical data, but, in other embodiments, the processing system 101 may be any other type of computing system operable to process medical sensing data. In the embodiments in which processing system 101 is a computer workstation, the system includes at least a processor such as a microcontroller or a dedicated central processing unit (CPU), a non-transitory computer-readable storage medium such as a hard drive, random access memory (RAM), and/or compact disk read only memory (CD-ROM), one or more video controllers such as a graphics processing unit (GPU), and a network communication device such as an Ethernet controller. In that regard, in some particular instances the processing system 101 is programmed to execute steps associated with the data acquisition, analysis, and co-registration described herein. Accordingly, it is understood that any steps related to data acquisition, data processing, instrument control, and/or other processing or control aspects of the present disclosure may be implemented by the processing system using corresponding instructions stored on or in a non-transitory computer readable medium accessible by the processing system. In some instances, the processing system 101 is a console computing device. In some particular instances, the processing system 101 is similar to the s5™ Imaging System or the s5i™ Imaging System, each available from Volcano Corporation. In some instances, the processing system 101 is portable (e.g., handheld, on a rolling cart, etc.). Further, it is understood that in some instances processing system 101 comprises a plurality of computing devices. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

In the illustrated embodiment, the medical system 100 is deployed in a catheter lab 102 having a control room 104, with the processing system 101 being located in the control room. In other embodiments, the processing system 101 may be located elsewhere, such as in the catheter lab 102, in a centralized area in the medical facility, or at an off-site location. The catheter lab 102 includes a sterile field but its associated control room 104 may or may not be sterile depending on the requirements of a procedure and/or health care facility. The catheter lab and control room may be used to perform on a patient any number of medical procedures such as angiography, intravascular ultrasound (IVUS), virtual histology (VH), forward looking IVUS (FL-IVUS), intravascular photoacoustic (IVPA) imaging, a fractional flow reserve (FFR) determination, a coronary flow reserve (CFR) determination, optical coherence tomography (OCT), computed tomography (CT), intracardiac echocardiography (ICE), forward-looking ICE (FLICE), intravascular palpography, transesophageal ultrasound, or any other medical sensing modalities known in the art. Further, the catheter lab and control room may be used to perform one or more treatment or therapy procedures on a patient such as radiofrequency ablation (RFA), cryotherapy, atherectomy or any other medical treatment procedure known in the art. For example, in catheter lab 102 a patient 106 may be undergoing a multi-modality procedure either as a single procedure or in combination with one or more sensing procedures. In any case, the catheter lab 102 includes a plurality of medical instruments including medical sensing devices that may collect medical sensing data in various different medical sensing modalities from the patient 106.

Instruments 108 and 110 are medical sensing devices that may be utilized by a clinician to acquire medical sensing data about the patient 106. In a particular instance, the device 108 collects medical sensing data in one modality and the device 110 collects medical sensing data in a different modality. For instance, the instruments may each collect one of pressure, flow (velocity), images (including images obtained using ultrasound (e.g., IVUS), OCT, thermal, and/or other imaging techniques), temperature, and/or combinations thereof. The instruments 108 and 110 may be any form of device, instrument, or probe sized and shaped to be positioned within a vessel, attached to an exterior of the patient, or scanned across a patient at a distance.

In the illustrated embodiment of FIG. 1, instrument 108 is an intravascular IVUS catheter that may include one or more sensors such as a phased-array transducer to collect IVUS sensing data. In some embodiments, the IVUS catheter may be capable of multi-modality sensing such as IVUS and IVPA sensing. Further, in the illustrated embodiment, the instrument 110 is an intravascular OCT catheter that may include one or more optical sensors configured to collect OCT sensing data. In some instances, an IVUS patient interface module (PIM) 112 and an OCT PIM 114 respectively couple the instrument 108 and instrument 110 to the medical system 100. In particular, the IVUS PIM 112 and the OCT PIM 114 are operable to respectively receive medical sensing data collected from the patient 106 by the instrument 108 and instrument 110 and are operable to transmit the received data to the processing system 101 in the control room 104. In one embodiment, the PIMs 112 and 114 include analog to digital (A/D) converters and transmit digital data to the processing system 101, however, in other embodiments, the PIMs transmit analog data to the processing system. In one embodiment, the IVUS PIM 112 and OCT PIM 114 transmit the medical sensing data over a Peripheral Component Interconnect Express (PCIe) data bus connection, but, in other embodiments, they may transmit data over a USB connection, a Thunderbolt connection, a FireWire connection, or some other high-speed data bus connection. In other instances, the PIMs may be connected to the processing system 101 via wireless connections using IEEE 802.11 Wi-Fi standards, Ultra Wide-Band (UWB) standards, wireless FireWire, wireless USB, or another high-speed wireless networking standard.

Additionally, in the medical system 100, an electrocardiogram (ECG) device 116 is operable to transmit electrocardiogram signals or other hemodynamic data from patient 106 to the processing system 101. In some embodiments, the processing system 101 may be operable to synchronize data collected with the instruments 108 and 110 using ECG signals from the ECG 116. Further, an angiogram system 117 is operable to collect x-ray, computed tomography (CT), or magnetic resonance images (MRI) of the patient 106 and transmit them to the processing system 101. In one embodiment, the angiogram system 117 may be communicatively coupled to the processing system to the processing system 101 through an adapter device. Such an adaptor device may transform data from a proprietary third-party format into a format usable by the processing system 101. In some embodiments, the processing system 101 may be operable to co-register image data from angiogram system 117 (e.g., x-ray data, MRI data, CT data, etc.) with sensing data from the instruments 108 and 110. As one aspect of this, the co-registration may be performed to generate three-dimensional images with the sensing data. In another embodiment, medical data from the ECG and/or angiogram system 117 may be temporally co-registered with medical data captured by either of (or both) instruments 108 and 110. Temporal co-registration using diagnostic windows will be discussed in greater detail in association with FIGS. 8-34.

A bedside controller 118 is also communicatively coupled to the processing system 101 and provides user control of the particular medical modality (or modalities) being used to diagnose the patient 106. In the current embodiment, the bedside controller 118 is a touch screen controller that provides user controls and diagnostic images on a single surface. In alternative embodiments, however, the bedside controller 118 may include both a non-interactive display and separate controls such as physical buttons and/or a joystick. In the integrated medical system 100, the bedside controller 118 is operable to present workflow control options and patient image data in graphical user interfaces (GUIs). The bedside controller 118 is capable displaying workflows and diagnostic images for multiple modalities allowing a clinician to control the acquisition of multi-modality medical sensing data with a single interface device.

A main controller 120 in the control room 104 is also communicatively coupled to the processing system 101 and, as shown in FIG. 1, is adjacent to catheter lab 102. In the current embodiment, the main controller 120 is similar to the bedside controller 118 in that it includes a touch screen and is operable to display multitude of GUI-based workflows corresponding to different medical sensing modalities via a UI framework service executing thereon. In some embodiments, the main controller 120 may be used to simultaneously carry out a different aspect of a procedure's workflow than the bedside controller 118. In alternative embodiments, the main controller 120 may include a non-interactive display and standalone controls such as a mouse and keyboard.

The medical system 100 further includes a boom display 122 communicatively coupled to the processing system 101. The boom display 122 may include an array of monitors, each capable of displaying different information associated with a medical sensing procedure. For example, during an IVUS procedure, one monitor in the boom display 122 may display a tomographic view and one monitor may display a sagittal view.

Further, in some embodiments, the multi-modality processing system 101 is communicatively coupled to a data network such as a TCP/IP-based local area network (LAN), a Synchronous Optical Networking (SONET) network, or a wide area network (WAN) or the Internet. The processing system 101 may connect to various resources via such a network. For example, the processing system 101 may communicate with a Digital Imaging and Communications in Medicine (DICOM) system, a Picture Archiving and Communication System (PACS), and a Hospital Information System (HIS) through the network.

Additionally, in the illustrated embodiment, medical instruments in system 100 discussed above are shown as communicatively coupled to the processing system 101 via a wired connection such as a standard copper link or a fiber optic link, but, in alternative embodiments, the tools may be connected to the processing system 101 via wireless connections using IEEE 802.11 Wi-Fi standards, Ultra Wide-Band (UWB) standards, wireless FireWire, wireless USB, or another high-speed wireless networking standard.

One of ordinary skill in the art would recognize that the medical system 100 described above is simply an example embodiment of a system that is operable to collect diagnostic data associated with a plurality of medical modalities. In alternative embodiments, different and/or additional tools may be communicatively coupled to the processing system 101 so as to contribute additional and/or different functionality to the medical system 100.

Referring to FIG. 2, shown therein is a vessel 124 having a stenosis according to an embodiment of the present disclosure. In that regard, FIG. 2 is a diagrammatic perspective view of the vessel 124 that includes a proximal portion 125 and a distal portion 126. A lumen 127 extends along the length of the vessel 124 between the proximal portion 125 and the distal portion 126. In that regard, the lumen 127 is configured to allow the flow of fluid through the vessel. In some instances, the vessel 124 is a systemic blood vessel. In some particular instances, the vessel 124 is a coronary artery. In such instances, the lumen 127 is configured to facilitate the flow of blood through the vessel 124.

As shown, the vessel 124 includes a stenosis 128 between the proximal portion 125 and the distal portion 126. Stenosis 128 is generally representative of any blockage or other structural arrangement that results in a restriction to the flow of fluid through the lumen 127 of the vessel 124. Embodiments of the present disclosure are suitable for use in a wide variety of vascular applications, including without limitation coronary, peripheral (including but not limited to lower limb, carotid, and neurovascular), renal, and/or venous. Where the vessel 124 is a blood vessel, the stenosis 128 may be a result of plaque buildup, including without limitation plaque components such as fibrous, fibro-lipidic (fibro fatty), necrotic core, calcified (dense calcium), blood, fresh thrombus, and mature thrombus. Generally, the composition of the stenosis will depend on the type of vessel being evaluated. In that regard, it is understood that the concepts of the present disclosure are applicable to virtually any type of blockage or other narrowing of a vessel that results in decreased fluid flow.

Note that the stenosis 128 is exemplary in nature and should be considered limiting in any way. In that regard, it is understood that the stenosis 128 has other shapes and/or compositions that limit the flow of fluid through the lumen 127 in other instances. While the vessel 124 is illustrated in FIG. 2 as having a single stenosis 128 and the description of the embodiments below is primarily made in the context of a single stenosis, it is nevertheless understood that the devices, systems, and methods described herein have similar application for a vessel having multiple stenosis regions.

Referring now to FIG. 3, illustrated is a partial cross-sectional perspective view of a portion of the vessel 124 taken along section line 3-3 of FIG. 2. In particular, the vessel 124 is shown with instruments 130 and 132 positioned therein according to an embodiment of the present disclosure. In general, instruments 130 and 132 may be any form of device, instrument, or probe sized and shaped to be positioned within a vessel. In that regard, in some instances instrument 132 is suitable for use as at least one of instruments 108 and 110 discussed above. Accordingly, in some instances the instrument 132 includes features similar to those discussed above with respect to instruments 108 and 110 in some instances. In the illustrated embodiment, instrument 130 is generally representative of a guide wire, while instrument 132 is generally representative of a catheter. In that regard, instrument 130 extends through a central lumen of instrument 132. However, in other embodiments, the instruments 130 and 132 take other forms. In that regard, the instruments 130 and 132 are of similar form in some embodiments. For example, in some instances, both instruments 130 and 132 are guide wires. In other instances, both instruments 130 and 132 are catheters. On the other hand, the instruments 130 and 132 are of different form in some embodiments, such as the illustrated embodiment, where one of the instruments is a catheter and the other is a guide wire. Further, in some instances, the instruments 130 and 132 are disposed coaxial with one another, as shown in the illustrated embodiment of FIG. 3. In other instances, one of the instruments extends through an off-center lumen of the other instrument. In yet other instances, the instruments 130 and 132 extend side-by-side. In some particular embodiments, at least one of the instruments is as a rapid-exchange device, such as a rapid-exchange catheter. In such embodiments, the other instrument is a buddy wire or other device configured to facilitate the introduction and removal of the rapid-exchange device. Further still, in other instances, instead of two separate instruments 130 and 132 a single instrument is utilized. In that regard, the single instrument incorporates aspects of the functionalities (e.g., data acquisition) of both instruments 130 and 132 in some embodiments.

Instrument 130 is configured to obtain medical diagnostic information (data) about the vessel 124. In that regard, the instrument 130 includes one or more sensors, transducers, and/or other monitoring elements configured to obtain the diagnostic information about the vessel. The diagnostic information includes one or more of pressure, flow (velocity), images (including images obtained using ultrasound (e.g., IVUS), OCT, thermal, and/or other imaging techniques), temperature, and/or combinations thereof. The one or more sensors, transducers, and/or other monitoring elements are positioned adjacent a distal portion of the instrument 130 in some instances. In that regard, the one or more sensors, transducers, and/or other monitoring elements are positioned less than 30 cm, less than 10 cm, less than 5 cm, less than 3 cm, less than 2 cm, and/or less than 1 cm from a distal tip 134 of the instrument 130 in some instances. In some instances, at least one of the one or more sensors, transducers, and/or other monitoring elements is positioned at the distal tip of the instrument 130.

The instrument 130 includes at least one element configured to monitor pressure within the vessel 124. The pressure monitoring element can take the form a piezo-resistive pressure sensor, a piezo-electric pressure sensor, a capacitive pressure sensor, an electromagnetic pressure sensor, a fluid column (the fluid column being in communication with a fluid column sensor that is separate from the instrument and/or positioned at a portion of the instrument proximal of the fluid column), an optical pressure sensor, and/or combinations thereof. In some instances, one or more features of the pressure monitoring element are implemented as a solid-state component manufactured using semiconductor and/or other suitable manufacturing techniques. Examples of commercially available guide wire products that include suitable pressure monitoring elements include, without limitation, the PrimeWire PRESTIGE® pressure guide wire, the PrimeWire® pressure guide wire, and the ComboWire® XT pressure and flow guide wire, each available from Volcano Corporation, as well as the PressureWire® Certus guide wire and the PressureWire™ Aeris guide wire, each available from St. Jude Medical, Inc. Generally, the instrument 130 is sized such that it can be positioned through the stenosis 128 without significantly impacting fluid flow across the stenosis, which would impact the distal pressure reading. Accordingly, in some instances the instrument 130 has an outer diameter of 0.018″ or less. In some embodiments, the instrument 130 has an outer diameter of 0.014″ or less.

Instrument 132 is also configured to obtain diagnostic information about the vessel 124. In some instances, instrument 132 is configured to obtain the same diagnostic information as instrument 130. In other instances, instrument 132 is configured to obtain different diagnostic information than instrument 130, which may include additional diagnostic information, less diagnostic information, and/or alternative diagnostic information. The medical diagnostic information (data) obtained by instrument 132 includes one or more of pressure, flow (velocity), images (including images obtained using ultrasound (e.g., IVUS), OCT, thermal, and/or other imaging techniques), temperature, and/or combinations thereof. Instrument 132 includes one or more sensors, transducers, and/or other monitoring elements configured to obtain this diagnostic information. In that regard, the one or more sensors, transducers, and/or other monitoring elements are positioned adjacent a distal portion of the instrument 132 in some instances. In that regard, the one or more sensors, transducers, and/or other monitoring elements are positioned less than 30 cm, less than 10 cm, less than 5 cm, less than 3 cm, less than 2 cm, and/or less than 1 cm from a distal tip 136 of the instrument 132 in some instances. In some instances, at least one of the one or more sensors, transducers, and/or other monitoring elements is positioned at the distal tip of the instrument 132.

Similar to instrument 130, instrument 132 also includes at least one element configured to monitor pressure within the vessel 124. The pressure monitoring element can take the form a piezo-resistive pressure sensor, a piezo-electric pressure sensor, a capacitive pressure sensor, an electromagnetic pressure sensor, a fluid column (the fluid column being in communication with a fluid column sensor that is separate from the instrument and/or positioned at a portion of the instrument proximal of the fluid column), an optical pressure sensor, and/or combinations thereof. In some instances, one or more features of the pressure monitoring element are implemented as a solid-state component manufactured using semiconductor and/or other suitable manufacturing techniques. Millar catheters are utilized in some embodiments. Currently available catheter products suitable for use with one or more of Philips's Xper Flex Cardio Physiomonitoring System, GE's Mac-Lab XT and XTi hemodynamic recording systems, Siemens's AXIOM Sensis XP VC11, McKesson's Horizon Cardiology Hemo, and Mennen's Horizon XVu Hemodynamic Monitoring System and include pressure monitoring elements can be utilized for instrument 132 in some instances.

In accordance with aspects of the present disclosure, at least one of the instruments 130 and 132 is configured to monitor a pressure within the vessel 124 distal of the stenosis 128 and at least one of the instruments 130 and 132 is configured to monitor a pressure within the vessel proximal of the stenosis. In that regard, the instruments 130, 132 are sized and shaped to allow positioning of the at least one element configured to monitor pressure within the vessel 124 to be positioned proximal and/or distal of the stenosis 128 as necessary based on the configuration of the devices. In that regard, FIG. 3 illustrates a position 138 suitable for measuring pressure distal of the stenosis 128. In that regard, the position 138 is less than 5 cm, less than 3 cm, less than 2 cm, less than 1 cm, less than 5 mm, and/or less than 2.5 mm from the distal end of the stenosis 128 (as shown in FIG. 2) in some instances. FIG. 3 also illustrates a plurality of suitable positions for measuring pressure proximal of the stenosis 128. In that regard, positions 140, 142, 144, 146, and 148 each represent a position that is suitable for monitoring the pressure proximal of the stenosis in some instances. In that regard, the positions 140, 142, 144, 146, and 148 are positioned at varying distances from the proximal end of the stenosis 128 ranging from more than 20 cm down to about 5 mm or less. Generally, the proximal pressure measurement will be spaced from the proximal end of the stenosis. Accordingly, in some instances, the proximal pressure measurement is taken at a distance equal to or greater than an inner diameter of the lumen of the vessel from the proximal end of the stenosis. In the context of coronary artery pressure measurements, the proximal pressure measurement is generally taken at a position proximal of the stenosis and distal of the aorta, within a proximal portion of the vessel. However, in some particular instances of coronary artery pressure measurements, the proximal pressure measurement is taken from a location inside the aorta. In other instances, the proximal pressure measurement is taken at the root or ostium of the coronary artery.

In one embodiment, the instrument 132 includes both a pressure sensor and an IVUS sensor. In such an embodiment, the plurality of sensors disposed on the instruments 130 and 132 may be utilized to perform a multi-modality diagnostic and/or treatment procedure. For example, the pressure sensor disposed on instrument 130 and the pressure sensor disposed on instrument 132 may collect medical data for an FFR calculation, and the IVUS sensor disposed on instrument 132 may collect medical data to be processed into IVUS images. As will be discussed below in greater detail, the collection of the pressure data and the IVUS data may be synchronized using heartbeat data from the patient using a heart-monitoring device such as the pressure sensors disposed on either of the instruments 130 and 132 or the ECG system 116 (FIG. 1). In one embodiment, a diagnostic window within the cardiac cycle of a patient is identified and collection of the pressure data and the IVUS data within the vessel 124 may occur exclusively within the diagnostic window. Such synchronization techniques may be applied to any type of collected medical data discussed herein, including OCT data, angiogram data, FFR data, IVUS data, VH data, FL-IVUS data, IVPA data, CFR data, CT data, ICE data, FLICE data, intravascular palpography data, transesophageal ultrasound data, or any other medical data known in the art. For instance, an intravascular image (e.g., IVUS, OCT) may be captured during an identified diagnostic window within a cardiac cycle of a patient, and an angiogram image (or other externally-captured image) may also be captured during the same diagnostic window, such that the images may be accurately temporally co-registered.

Additionally, the instruments 130 and 132 may be sized and shaped to allow concurrent positioning of additional catheter-type instruments within the vessel 124. For instance, in one embodiment, the instrument 108 may have a greater diameter than the instrument 132, permitting it to slide over both instruments 130 and 132 and into a position near the stenosis 128. Once positioned, the instrument 108 may collect medical data associated with the vessel 124 using an IVUS sensor. During a multi-modality procedure, the collection of IVUS data may occur concurrently or subsequently to any collection of data with instruments 130 and 132. As mentioned above, synchronization of such data collection may be carried out using heartbeat data in the form of pressure data, flow data, and/or ECG data. It is understood that the instruments 130 and 132 may be sized and shaped to allow any number of additional instruments to be positioned within the vessel 124 during a multi-modality procedure.

Referring now to FIG. 4, illustrated is a diagrammatic, schematic view of portions of the medical system 100 according to aspects of the present disclosure. As shown, in addition to the instruments 108 and 110, the medical system 100 includes an instrument 152. In that regard, in some instances instrument 152 is suitable for use as at least one of instruments 130 and 132 discussed above. Accordingly, in some instances the instrument 152 includes features similar to those discussed above with respect to instruments 130 and 132 in some instances. In the illustrated embodiment, the instrument 152 is a guide wire having a distal portion 154 and a housing 156 positioned adjacent the distal portion. In that regard, the housing 156 is spaced approximately 3 cm from a distal tip of the instrument 152. The housing 156 is configured to house one or more sensors, transducers, and/or other monitoring elements configured to obtain the diagnostic information about the vessel. In the illustrated embodiment, the housing 156 contains at least a pressure sensor configured to monitor a pressure within a lumen in which the instrument 152 is positioned. A shaft 158 extends proximally from the housing 156. A torque device 160 is positioned over and coupled to a proximal portion of the shaft 158. A proximal end portion 162 of the instrument 152 is coupled to a connector 164. A cable 166 extends from connector 164 to a connector 168. In some instances, connector 168 is configured to be plugged into an interface 170. In that regard, interface 170 is a patient interface module (PIM) in some instances. In some instances, the cable 166 is replaced with a wireless connection. In that regard, it is understood that various communication pathways between the instrument 152 and the interface 170 may be utilized, including physical connections (including electrical, optical, and/or fluid connections), wireless connections, and/or combinations thereof. The interface 170 is communicatively coupled to the multi-modality processing system 101 via a connection 174.

Together, connector 164, cable 166, connector 168, interface 170, and connection 174 facilitate communication between the one or more sensors, transducers, and/or other monitoring elements of the instrument 152 and the processing system 101. However, this communication pathway is exemplary in nature and should not be considered limiting in any way. In that regard, it is understood that any communication pathway between the instrument 152 and the processing system 101 may be utilized, including physical connections (including electrical, optical, and/or fluid connections), wireless connections, and/or combinations thereof. In that regard, it is understood that the connection 174 is wireless in some instances. In some instances, the connection 174 includes a communication link over a network (e.g., intranet, internet, telecommunications network, and/or other network). In that regard, it is understood that the processing system 101 is positioned remote from an operating area where the instrument 152 is being used in some instances. Having the connection 174 include a connection over a network can facilitate communication between the instrument 152 and the remote processing system 101 regardless of whether the processing system is in an adjacent room, an adjacent building, or in a different state/country. Further, it is understood that the communication pathway between the instrument 152 and the processing system 101 is a secure connection in some instances. Further still, it is understood that, in some instances, the data communicated over one or more portions of the communication pathway between the instrument 152 and the processing system 101 is encrypted.

The medical system 100 also includes an instrument 175. In that regard, in some instances instrument 175 is suitable for use as at least one of instruments 130 and 132 discussed above. Accordingly, in some instances the instrument 175 includes features similar to those discussed above with respect to instruments 130 and 132 in some instances. In the illustrated embodiment, the instrument 175 is a catheter-type device. In that regard, the instrument 175 includes one or more sensors, transducers, and/or other monitoring elements adjacent a distal portion of the instrument configured to obtain the diagnostic information about the vessel. In the illustrated embodiment, the instrument 175 includes a pressure sensor configured to monitor a pressure within a lumen in which the instrument 175 is positioned. The instrument 175 is in communication with an interface 176 via connection 177. In some instances, interface 176 is a hemodynamic monitoring system or other control device, such as Siemens AXIOM Sensis, Mennen Horizon XVu, and Philips Xper IM Physiomonitoring 5. In one particular embodiment, instrument 175 is a pressure-sensing catheter that includes fluid column extending along its length. In such an embodiment, interface 176 includes a hemostasis valve fluidly coupled to the fluid column of the catheter, a manifold fluidly coupled to the hemostasis valve, and tubing extending between the components as necessary to fluidly couple the components. In that regard, the fluid column of the catheter is in fluid communication with a pressure sensor via the valve, manifold, and tubing. In some instances, the pressure sensor is part of interface 176. In other instances, the pressure sensor is a separate component positioned between the instrument 175 and the interface 176. The interface 176 is communicatively coupled to the processing system 101 via a connection 178.

Similar to the connections between instrument 152 and the processing system 101, interface 176 and connections 177 and 178 facilitate communication between the one or more sensors, transducers, and/or other monitoring elements of the instrument 175 and the processing system 101. However, this communication pathway is exemplary in nature and should not be considered limiting in any way. In that regard, it is understood that any communication pathway between the instrument 175 and the processing system 101 may be utilized, including physical connections (including electrical, optical, and/or fluid connections), wireless connections, and/or combinations thereof. In that regard, it is understood that the connection 178 is wireless in some instances. In some instances, the connection 178 includes a communication link over a network (e.g., intranet, internet, telecommunications network, and/or other network). In that regard, it is understood that the processing system 101 is positioned remote from an operating area where the instrument 175 is being used in some instances. Having the connection 178 include a connection over a network can facilitate communication between the instrument 175 and the remote processing system 101 regardless of whether the processing system is in an adjacent room, an adjacent building, or in a different state/country. Further, it is understood that the communication pathway between the instrument 175 and the processing system 101 is a secure connection in some instances. Further still, it is understood that, in some instances, the data communicated over one or more portions of the communication pathway between the instrument 175 and the processing system 101 is encrypted.

It is understood that one or more components of the medical system 100 are not included, are implemented in a different arrangement/order, and/or are replaced with an alternative device/mechanism in other embodiments of the present disclosure. For example, in some instances, the medical system 100 does not include interface 170 and/or interface 176. In such instances, the connector 168 (or other similar connector in communication with instrument 152 or instrument 175) may plug into a port associated with processing system 101. Alternatively, the instruments 152, 175 may communicate wirelessly with the processing system 101. Generally speaking, the communication pathway between either or both of the instruments 152, 175 and the processing system 101 may have no intermediate nodes (i.e., a direct connection), one intermediate node between the instrument and the processing system, or a plurality of intermediate nodes between the instrument and the processing system.

Additionally, the instruments 152 and 175 are sized and shaped to allow concurrent positioning of additional catheter-type instruments within a vessel. For instance, in one embodiment, the instrument 108 may have a greater diameter than the instrument 175, permitting it to slide over both instruments 152 and 175 and into a position near a position of interest within a vessel. In turn, the instrument 110 may have a diameter greater than the instrument 108, permitting it to slide over instruments 108, 152 and 175. Once positioned, the instruments 108 and 110 may collect medical data using IVUS and OCT sensors concurrently or subsequently to any collection of data with instruments 130 and 132. During such a multi-modality procedure, co-registration of data collection may be carried out using heartbeat data in the form of pressure data, flow data, and/or ECG data. For instance, data collection by each of the medical instruments may be coordinated to occur during a diagnostic window within the cardiac cycle of the patient. In that regard, the figures below illustrate various manners in which to identify diagnostic windows for synchronization of multi-modality data collection.

Referring now to FIGS. 5-8, shown therein are graphical representations of diagnostic information illustrating aspects of an embodiment of the present disclosure. In that regard, FIG. 5 is a graphical representation of measured pressure, velocity, and resistance within a vessel; FIG. 6 is a magnified view of a portion of the graphical representation of FIG. 5 corresponding to a resting state of a patient; FIG. 7 is a magnified view of a portion of the graphical representation of FIG. 5 corresponding to a hyperemic state of a patient; and FIG. 8 is the portion of the graphical representation of FIG. 6 annotated to identify a diagnostic window according to an embodiment of the present disclosure.

Referring more particularly to FIG. 5, shown therein is a graphical representation 180 of diagnostic information pertaining to a vessel. More specifically, the graphical representation 180 includes a graph 182 plotting pressure within the vessel over time, a graph 184 plotting velocity of the fluid within the vessel over time, and a graph 186 plotting resistance within the vessel over time. In that regard, the resistance (or impedance) shown in graph 186 is calculated based on the pressure and velocity data of graphs 182 and 184. In particular, the resistance values shown in graph 186 are determined by dividing the pressure measurement of graph 182 by the velocity measurement 184 for the corresponding point in time. The graphical representation 180 includes a time period 188 that corresponds to a resting state of the patient's heart and a time period 190 that corresponds to a stressed state of the patient's heart.

To better illustrate the differences in the pressure, velocity, and resistance data between the resting and stressed states of the patient, close-up views of the data within windows 192 and 194 are provided in FIGS. 6 and 7. Referring more specifically to FIG. 6, window 192 of the graphical representation 180 includes graph portions 196, 198, and 200 that correspond to graphs 182, 184, and 186, respectively. As shown, in the resting state of FIG. 6, the resistance within the vessel has an average value of approximately 0.35 on the scale of graph 200, as indicated by line 202. Referring now to FIG. 7, window 194 of the graphical representation 180 includes graph portions 204, 206, and 208 that correspond to graphs 182, 184, and 186, respectively. As shown, in the stressed state of FIG. 7, the resistance within the vessel is significantly less than the resting state with a value of approximately 0.20 on the scale of graph 208, as indicated by line 210.

Referring to FIG. 8, similar to FIG. 6 window 192 of the graphical representation 180 of FIG. 5 is shown and includes graph portions 196, 198, and 200 that correspond to graphs 182, 184, and 186, respectively. However, in FIG. 8 a section 212 of the heartbeat cycle of the patient has been identified. As shown, section 212 corresponds to the portion of the heartbeat cycle of the patient where the resistance is reduced without the use of a hyperemic agent or other stressing technique. That is, section 212 is a portion of the heartbeat cycle of a resting patient that has a naturally reduced and relatively constant resistance. In other instances, section 212 of the heartbeat cycle encompasses the portion the heartbeat cycle that is less than a fixed percentage of the maximum resistance of the heartbeat cycle. In that regard, the fixed percentage of the maximum resistance of the heartbeat cycle is less than 50%, less than 30%, less than 25%, less than 20%, less than 15%, less than 10%, and less than 5% in some embodiments. In yet other instances, section 212 of the heartbeat cycle encompasses the portion the heartbeat cycle that is less than a fixed percentage of the average resistance of the heartbeat cycle. In that regard, the fixed percentage of the average resistance of the heartbeat cycle is less than 75%, less than 50%, less than 25%, less than 20%, less than 15%, less than 10%, and less than 5% in some embodiments.

Accordingly, in some embodiments of the present disclosure, the portion of the heartbeat cycle coinciding with section 212 is utilized as a diagnostic window for temporally synchronizing data collection during a multi-modality procedure. That is, medical data in first modality will be collected during the diagnostic window and medical data in a second, different modality will also be collected during the diagnostic window. In some instances, medical data corresponding to all selected modalities will be collected during the same diagnostic window within the same cardiac cycle. In other instances, medical data corresponding to a first modality will be collected during a diagnostic window in a first cardiac cycle and medical data corresponding to a second modality will be collected during the same diagnostic window but in a subsequent cardiac cycle. Multi-modality data collection within a diagnostic window will be discussed in greater detail in association with FIGS. 31-34.

In this manner, all medical data collected across the modalities will be representative of the same portion of a patient's heartbeat cycle. As a result, resultant co-registered patient data may be more useful and/or accurate during a diagnosis by a health care provider. Additionally, by using a window of non-trivial length rather than an instantaneous point for the synchronization of data collection, data collection systems need not be as accurate in their data sampling. Reduction in accuracy requirements may reduce costs and complexity. Further, in multi-modality procedures designed to evaluate a stenosis in a coronary artery or other vessel with dramatic fluctuations in resistance, the diagnostic windows identified by the methods herein are portions of a patient's heartbeat cycle that have a naturally reduced and relatively constant resistance. Accordingly, multi-modality evaluation of a stenosis of the vessel is possible without the use of a hyperemic agent or other stressing of the patient's heart. Use of such diagnostic windows may be especially advantageous if a multi-modality procedure includes an FFR evaluation. In such an embodiment, to compute an FFR value, the pressure ratio (distal pressure divided by proximal pressure) across the stenosis is calculated for the time period corresponding to section 212 for one or more heartbeats. The calculated pressure ratio is an average over the diagnostic window defined by section 212 in some instances. By comparing the calculated pressure ratio to a threshold or predetermined value in combination with concurrently collected data in a different modality, a physician or other treating medical personnel can determine what, if any, treatment should be administered.

In some instances, section 212 is identified by monitoring pressure and fluid flow velocity within the vessel using one or more instruments and calculating the resistance within the vessel based on the measured pressure and velocity. For example, referring again to the embodiment of FIG. 3, in some instances the instrument 130 includes one or more sensing elements configured to monitor at least pressure and flow velocity, while instrument 132 includes one or more sensing elements configured to monitor at least pressure. Accordingly, with the one or more sensing elements of instrument 130 positioned distal of the stenosis and the one or more sensing elements of instrument 132 positioned proximal of the stenosis, the pressure and flow velocity measurements obtained by instrument 130 are utilized to identify section 212.

In other instances, section 212 is identified without monitoring fluid velocity. In that regard, several techniques for identifying suitable diagnostic windows for use in multi-modality data collection procedures are described below. In some instances, the diagnostic window is identified solely based on characteristics of the pressure measurements obtained by instruments positioned within the vessel. Accordingly, in such instances, the instruments utilized need only have elements configured to monitor a pressure within the vessel, which results in reduced cost and simplification of the system. Exemplary techniques for evaluating a vessel based on pressure measurements are described in UK Patent Application No. 1100137.7 filed Jan. 6, 2011 and titled “APPARATUS AND METHOD OF ASSESSING A NARROWING IN A FLUID FILLED TUBE”, which is hereby incorporated by reference in its entirety.

In general, the diagnostic window may be identified based on characteristics and/or components of one or more of proximal pressure measurements, distal pressure measurements, proximal velocity measurements, distal velocity measurements, ECG waveforms, and/or other identifiable and/or measurable aspects of vessel performance. In that regard, various signal processing and/or computational techniques can be applied to the characteristics and/or components of one or more of proximal pressure measurements, distal pressure measurements, proximal velocity measurements, distal velocity measurements, ECG waveforms, and/or other identifiable and/or measurable aspects of vessel performance to identify a suitable diagnostic window.

In some embodiments, the determination of the diagnostic window and/or the multi-modality data collection are performed in approximately real time or live to identify the section 212 and collect the multi-modality data. In that regard, collecting the multi-modality data in “real time” or “live” within the context of the present disclosure is understood to encompass calculations that occur within 10 seconds of data acquisition. It is recognized, however, that often “real time” or “live” calculations are performed within 1 second of data acquisition. In some instances, the “real time” or “live” calculations are performed concurrent with data acquisition. In some instances the calculations are performed by a processor in the delays between data acquisitions. For example, if data is acquired from the pressure sensing devices for 1 ms every 5 ms, then in the 4 ms between data acquisitions the processor can perform the calculations. It is understood that these timings are for example only and that data acquisition rates, processing times, and/or other parameters surrounding the calculations will vary. For example, in some embodiments, the data utilized to identify the diagnostic window are stored for later analysis.

Referring now to FIGS. 9-11, shown therein are graphical representations of diagnostic information illustrating aspects of another embodiment of the present disclosure. In that regard, FIG. 9 is a graphical representation of measured pressure and velocity within a vessel; FIG. 10 is a graphical representation of a differential of the measured velocity of FIG. 9; and FIG. 11 is the graphical representation of measured pressure and velocity within the vessel annotated to identify a diagnostic window according to an embodiment of the present disclosure.

Referring more specifically to FIG. 9, graphical representation 220 includes a plot 222 representative of pressure (measured in mmHg) within a vessel over the time period of one cardiac cycle and a plot 224 representative of velocity (measured in m/s) of a fluid within the vessel over the same cardiac cycle. FIG. 10, in turn, is a graphical representation 230 of a differential of the velocity plot 224 of graphical representation 220 of FIG. 9. In that regard, in some instances, the velocity differential or change in velocity (dU) is calculated as

$$d {U}_{xy} = \frac{{U}_{x} - {U}_{y}}{t} ,$$

where Uₓ is the velocity at time x, U_{y} is the velocity at time y, and t is the elapsed time between Uₓ and U_{y}. In some instances, the variable t is equal to the sample rate of the velocity measurements of the system such that the differential is calculated for all data points. In other instances, the variable t is longer than the sample rate of the velocity measurements of the system such that only a subset of the obtained data points are utilized.

As shown in FIG. 10, for a time period 232 extending from about 625 ms to about 1000 ms the differential of the velocity plot 224 is relatively stabilized around zero. In other words, the velocity of the fluid within the vessel and/or the vascular resistance is relatively constant during time period 232. In some instances, the velocity is considered stabilized when it varies between −0.01 and +0.01, and in some specific instance is considered stabilized when it varies between about −0.005 and about +0.005. However, in other instances, the velocity is considered stabilized with values outside of these ranges. Similarly, for a time period 234 extending from about 200 ms to about 350 ms the differential of the velocity plot 224 is relatively stabilized around zero representing that the velocity of the fluid within the vessel is substantially constant during time period 234 as well. However, time period 234 can be highly variable, as valvular disease, dyssynchrony within a ventricle, regional myocardial contractile differences, microvascular disease can all lead to large variations of timing of the time period 234. As discussed below, all or portions of the time periods 232 and/or 234 are utilized as a diagnostic window for the synchronization of multi-modality data collection in some embodiments of the present disclosure. In that regard, the diagnostic window is selected by identifying a portion of the cardiac cycle corresponding to the time period in which the change in velocity (i.e., dU) fluctuates around zero. FIG. 11 shows the graphical representation 220 of FIG. 9 annotated to identify a diagnostic window 236 corresponding to the time period 232 of FIG. 10. In other instances, the diagnostic window is selected by identifying a portion of the cardiac cycle corresponding to a period in which the change in velocity (i.e., dU) is relatively small compared to the maximum change in velocity (i.e., dUₘₐₓ) during a cardiac cycle. In the illustrated embodiment of FIG. 10, the maximum change in velocity (i.e., dUₘₐₓ) occurs at point 235. In some instances, the diagnostic window is selected by identifying the portion(s) of the cardiac cycle where the change in velocity (i.e., dU) is less than 25%, less than 20%, less than 15%, less than 10%, and/or less than 5% of the maximum change in velocity (i.e., dUₘₐₓ) for the cardiac cycle.

There are a variety of signal processing techniques that can be utilized to identify time period 232, time period 234, and/or other time periods where the change in velocity is relatively constant and approximately zero, such as variation or standard deviation from the mean, minimum threshold offset, or otherwise. Further, while time periods 232 and 234 have been identified using a differential of the velocity measurement, in other instances first, second, and/or third derivatives of the velocity measurement are utilized. For example, identifying time periods during the cardiac cycle where the first derivative of velocity is relatively constant and approximately zero allows the localization of time periods where velocity is relatively constant. Further, identifying time periods during the cardiac cycle where the second derivative of velocity is relatively constant and approximately zero allows the localization of a time period where acceleration is relatively constant and near zero, but not necessarily zero.

Time periods 232, 234, and/or other time periods where the change in velocity is relatively constant and approximately zero (i.e., the speed of the fluid flow is stabilized) are suitable diagnostic windows for the synchronization of multi-modality data collection in accordance with the present disclosure. In that regard, in a fluid flow system, the separated forward and backward generated pressures are defined by:

$$d {P}_{+} = \frac{1}{2} ( d P + ρ c d U ) and d {P}_{-} = \frac{1}{2} ( d P - ρ c d U ) ,$$

where dP is the differential of pressure, ρ is the density of the fluid within the vessel, c is the wave speed, and dU is the differential of flow velocity. However, where the flow velocity of the fluid is substantially constant, dU is approximately zero and the separated forward and backward generated pressures are defined by:

$$d {P}_{+} = \frac{1}{2} ( d P + ρ c ( 0 ) ) = \frac{1}{2} d P and d {P}_{-} = \frac{1}{2} ( d P - ρ c ( 0 ) ) = \frac{1}{2} d P .$$

In other words, during the time periods where dU is approximately zero, the forward and backward generated pressures are defined solely by changes in pressure.

Accordingly, during such time periods the severity of a stenosis within the vessel can be evaluated based on pressure measurements taken proximal and distal of the stenosis. In that regard, by comparing the forward and/or backward generated pressure distal of a stenosis to the forward and/or backward generated pressure proximal of the stenosis, an evaluation of the severity of the stenosis can be made. For example, the forward-generated pressure differential can be calculated as

$$\frac{d {P}_{+ distal}}{d {P}_{+ proximal}} ,$$

while the backward-generated pressure differential can be calculated as

$$\frac{d {P}_{- distal}}{d {P}_{- proximal}} .$$

In the context of the coronary arteries, a forward-generated pressure differential is utilized to evaluate a stenosis in some instances. In that regard, the forward-generated pressure differential is calculated based on proximally originating (i.e., originating from the aorta) separated forward pressure waves and/or reflections of the proximally originating separated forward pressure waves from vascular structures distal of the aorta in some instances. In other instances, a backward-generated pressure differential is utilized in the context of the coronary arteries to evaluate a stenosis. In that regard, the backward-generated pressure differential is calculated based on distally originating (i.e., originating from the microvasculature) separated backward pressure waves and/or reflections of the distally originating separated backward pressure waves from vascular structures proximal of the microvasculature.

In yet other instances, a pressure wave is introduced into the vessel by an instrument or medical device. In that regard, the instrument or medical device is utilized to generate a proximally originating forward pressure wave, a distally originating backward pressure wave, and/or combinations thereof for use in evaluating the severity of the stenosis. For example, in some embodiments an instrument having a movable membrane is positioned within the vessel. The movable membrane of the instrument is then activated to cause movement of the membrane and generation of a corresponding pressure wave within the fluid of the vessel. Based on the configuration of the instrument, position of the membrane within the vessel, and/or the orientation of the membrane within the vessel the generated pressure wave(s) will be directed distally, proximally, and/or both. Pressure measurements based on the generated pressure wave(s) can then be analyzed to determine the severity of the stenosis.

Referring now to FIGS. 12-15, shown therein are graphical representations of diagnostic information illustrating aspects of another embodiment of the present disclosure. In that regard, FIG. 12 is a graphical representation of wave intensity within a vessel; FIG. 13 is a graphical representation of proximal and distal originating pressure waves within the vessel corresponding to the wave intensity of FIG. 12; FIG. 14 is a graphical representation of pressure and velocity within the vessel corresponding to the wave intensity of FIG. 12 and the proximal and distal originating pressure waves of FIG. 13; and FIG. 15 is a graphical representation of a resistance within the vessel corresponding to the wave intensity of FIG. 12, the proximal and distal originating pressure waves of FIG. 13, and the pressure and velocity of FIG. 14.

Referring more specifically to FIG. 12, shown therein is a graphical representation 240 plotting the intensities associated with proximally and distally originating waves of a cardiac cycle over time. In that regard, plot 242 is representative of proximally originating waves, while plot 244 is representative of distally originating waves. As shown, six predominating waves are associated with the cardiac cycle of a patient. In order of occurrence during a cardiac cycle, wave 246 is a backward-traveling pushing wave, wave 248 is a dominant forward-traveling pushing wave, wave 250 is a backward-traveling pushing wave, wave 252 is a forward-traveling suction wave, wave 254 is a dominant backward-traveling suction wave, and wave 256 is a forward-traveling pushing wave. Notably, no waves are generated during a time period 258 late in the cardiac cycle. In some instances, the time period 258 is referred to as a wave-free period of the cardiac cycle. Additional details regarding pressure waves in the context of the coronary arteries can be found in “Evidence of a Dominant Backward-Propagating ‘Suction’ Wave Responsible for Diastolic Coronary Filling in Humans, Attenuated in Left Ventricular Hypertrophy” by Davies et al. (Circulation. 2006; 113:1768-1778), which is hereby incorporated by reference in its entirety.

Referring now to FIG. 13, shown therein is a graphical representation 260 of proximal and distal originating pressure waves within a vessel over a time period associated with a cardiac cycle. In that regard, the pressure waves of FIG. 13 correspond to the wave intensities of FIG. 12. As shown, the graphical representation 260 includes a plot 262 representative of a proximally-originating pressure, a plot 264 representative of a distally-originating pressure, and a plot 265 representative of the total pressure (proximally-originating pressure plus the distally-originating pressure).

Referring now to FIG. 14, shown therein is a graphical representation 270 that includes a plot 272 representative of pressure (measured in mmHg) within a vessel over time and a plot 274 representative of velocity (measured in cm/s) of a fluid within the vessel over time. In that regard, the pressure and velocity plots 272, 274 of FIG. 14 correspond to the wave intensities and pressure waves of FIGS. 12 and 13, respectively. As shown, for the wave-free time period 258 extending from about 475 ms to about 675 ms the slopes of the pressure plot 272 and the velocity plot 274 are relatively constant. At this time point, as shown in FIG. 15, the resistance within the vessel is relatively constant and reduced during the time period 258. In that regard, the graphical representation 280 of FIG. 15 includes a plot 282 of the resistance within the vessel over the time of a cardiac cycle. In that regard, the resistance values of graphical representation 280 are calculated using the pressure and velocity measurements of FIG. 14, where resistance is equal to pressure divided by velocity for a particular point in time along the cardiac cycle. Due to the reduced and relative constant resistance during time period 258, all or a portion of the time period 258 is suitable for use as a diagnostic window for evaluating pressure differential across a stenosis in some embodiments of the present disclosure. In that regard, in some embodiments the diagnostic window is the period of minimum resistance that corresponds to the wave-free period at the end of the backward-travelling suction wave, running to shortly before the end of the cardiac cycle.

Referring now to FIGS. 16-26, shown therein are various graphical representations of techniques for determining start and/or end points for a diagnostic window in accordance with the present disclosure. In that regard, FIGS. 16-18 generally illustrate identification of a starting point of a diagnostic window based on a proximal pressure measurement; FIGS. 19-22 generally illustrate identification of a starting point of a diagnostic window based on a distal pressure measurement; FIG. 23 illustrates identification of an end of a diagnostic window based on a starting point of the diagnostic window; FIG. 24 illustrates identification of an ending point of a diagnostic window based on a proximal pressure measurement; and FIGS. 25 and 26 illustrate identification of an ending point of a diagnostic window based on a distal pressure measurement.

As shown in FIG. 16, a graphical representation 300 includes a proximal pressure reading 302 and a distal pressure reading 304 each plotted over time relative to a cardiac cycle. In that regard, the proximal pressure reading 302 is representative of a pressure proximal of a stenosis of a vessel. The proximal pressure reading 302 is based upon a partial pressure (e.g., forward generated or backward generated) in some instances. Similarly, the distal pressure reading 304 is representative of a pressure distal of the stenosis. The distal pressure reading 304 is based upon a partial pressure (e.g., forward generated or backward generated) in some instances.

For simplicity and consistency, the proximal and distal pressure readings 302 and 304 provided in FIG. 16 will be utilized in describing the techniques associated with FIGS. 17-28 as well. However, with respect to all of the disclosed techniques the proximal and distal pressure readings 302 and 304 are exemplary and should not be considered limiting in any way. In that regard, it is understood that the pressure readings will vary from patient to patient and even between cardiac cycles of a single patient. Accordingly, it is understood that the techniques described herein for identifying a diagnostic window based on these pressure readings are suitable for use with a wide variety of pressure reading plots. Further, it is understood that the techniques described below are calculated or determined over a plurality of cardiac cycles in some instances. For example, in some embodiments the diagnostic window is identified by making calculations over a plurality of cardiac cycles and calculating an average or mean value, identifying overlapping areas common to the plurality of cardiac cycles, and/or otherwise identifying a suitable time period for a diagnostic window. Further still, it is understood that two or more of the techniques described below may be utilized together to identify a starting point, ending point, and/or other aspect of a diagnostic window.

Referring now to FIGS. 16-18, shown therein are several techniques for identifying a starting point of a diagnostic window based on a proximal pressure measurement. Referring more specifically to FIG. 16, the starting point of the diagnostic window is determined by identifying a dicrotic notch and adding a fixed amount of time in some instances. As shown in FIG. 16, a dicrotic notch 306 has been identified and a fixed time period 308 has been added to determine the starting point 310 of a diagnostic window. The fixed time period 308 is between about 1 ms and about 500 ms in some instances. In some particular instances, the time period 308 is between about 25 ms and about 150 ms. In other instances, the amount of time added to the start of diastole is selected based on a percentage of the cardiac cycle or a percentage of the length of diastole. For example, in some instances, the amount of time added is between about 0% and about 70% of the length of the cardiac cycle. In yet other instances, no time is added to the dicrotic notch, such that the dicrotic notch 306 is the starting point 310.

In another embodiment, a start of diastole is identified based on the proximal pressure measurements and a fixed time period is added to determine the starting point of a diagnostic window. The fixed time period is between about 1 ms and about 500 ms. In some particular embodiments, the fixed time period is between the beginning of diastole and the start of the diagnostic window is between about 25 ms and about 200 ms. In other instances, the amount of time added to the start of diastole is selected based on a percentage of the cardiac cycle or a percentage of the length of diastole. For example, in some instances, the time added to the start of diastole is between about 0% and about 70% of the cardiac cycle. In other instances, the time added to the start of diastole is between about 0% and about 100% of the total length of the diastole portion of the cardiac cycle. In some instances, the time added to the start of diastole is between about 2% and about 75% of the total length of the diastole portion of the cardiac cycle. In yet other instances, no time is added to the start of diastole, such that the start of diastole is also the starting point of the diagnostic window.

Referring now to FIG. 17, the starting point of the diagnostic window is determined by identifying a peak proximal pressure and adding a fixed amount of time in some instances. As shown in the graphical representation 312 of FIG. 17, a peak pressure 314 has been identified and a fixed time period 316 has been added to determine the starting point 318 of a diagnostic window. The fixed time period 316 is between about 1 ms and about 550 ms in some instances. In some instances, the fixed time period 316 is between about 25 ms and about 175 ms. In other instances, the amount of time added to the peak proximal pressure is selected based on a percentage of the cardiac cycle or a percentage of the length of diastole. For example, in some instances, the amount of time added is between about 0% and about 70% of the length of the cardiac cycle. In yet other instances, no time is added to the peak proximal pressure, such that the peak pressure 314 is the starting point 318.

Referring now to FIG. 18, the starting point of the diagnostic window is determined by identifying the start of a cardiac cycle and adding a fixed amount of time in some instances. As shown in the graphical representation 320 of FIG. 18, a start 322 of the cardiac cycle has been identified and a fixed time period 324 has been added to determine the starting point 326 of a diagnostic window. The fixed time period 324 is between about 150 ms and about 900 ms in some instances. In some instances, the fixed time period 324 is between about 300 ms and about 600 ms. In some particular embodiments, the fixed time period 324 is calculated as a percentage of the length 328 of a cardiac cycle of the patient. As shown in FIG. 18, an end 330 of the cardiac cycle has been identified such that the length 328 of the cardiac cycle extends between the start 322 and the end 330. The percentage of the length 328 of the cardiac cycle utilized for calculating the starting point 356 is between about 25% and about 95% in some instances. In some instances, the percentage of the length 328 of the cardiac cycle is between about 40% and about 75%. In yet other instances, no time is added to the start of the cardiac cycle, such that the start of the cardiac cycle 322 is the starting point 326.

Referring now to FIGS. 19-22, shown therein are several techniques for identifying a starting point of a diagnostic window based on a distal pressure measurement. Referring more specifically to FIG. 19, the starting point of the diagnostic window is determined by identifying a dicrotic notch and adding a fixed amount of time in some instances. As shown in the graphical representation 332 of FIG. 19, a dicrotic notch 334 has been identified and a fixed time period 336 has been added to determine the starting point 338 of a diagnostic window. The fixed time period 336 is between about 1 ms and about 500 ms in some instances. In some instances, the fixed time period 336 is between about 25 ms and about 150 ms. In other instances, a peak pressure 339 is identified based on the distal pressure measurements and a fixed time period is added to determine the starting point of a diagnostic window. The fixed time period relative to the peak pressure is between about 1 ms and about 550 ms in some instances. In some instances, the fixed time period is between about 25 ms and about 175 ms. In yet other instances, no time is added to the dicrotic notch, such that the dicrotic notch 334 is the starting point 338.

In another embodiment, a start of diastole is identified based on the distal pressure measurements and a fixed time period is added to determine the starting point of a diagnostic window. The fixed time period is between about 1 ms and about 500 ms. In some particular embodiments, the fixed time period between the beginning of diastole and the start of the diagnostic window is between about 25 ms and about 200 ms. In other instances, the amount of time added to the start of diastole is selected based on a percentage of the cardiac cycle or a percentage of the length of diastole. For example, in some instances, the time added to the start of diastole is between about 0% and about 70% of the cardiac cycle. In other instances, the time added to the start of diastole is between about 0% and about 100% of the total length of the diastole portion of the cardiac cycle. In some instances, the time added to the start of diastole is between about 2% and about 75% of the total length of the diastole portion of the cardiac cycle. In yet other instances, no time is added to the start of diastole, such that the start of diastole is the starting point of the diagnostic window.

Referring now to FIG. 20, the starting point of the diagnostic window is determined by identifying a maximum change in pressure and adding a fixed amount of time in some instances. In some particular instances, the maximum change in pressure after a peak distal pressure is utilized as the basis point from which the fixed amount of time is added. As shown in the graphical representation 340 of FIG. 20, after peak pressure 342 the point having a maximum change in pressure (i.e., dP/dt) is identified by point 344. A fixed time period 346 has been added to point 344 to determine the starting point 348 of a diagnostic window. The fixed time period 346 is between about 1 ms and about 500 ms in some instances. In some instances, the fixed time period 346 is between about 25 ms and about 150 ms. In some particular embodiments, the fixed time period 346 is calculated as a percentage of the length of the cardiac cycle of the patient. The percentage of the length of the cardiac cycle utilized for calculating the starting point 348 is between about 0% and about 70% in some instances. In yet other instances, no time is added to the point 344 representative of the maximum change in pressure, such that the point 344 is the starting point 348.

Referring now to FIG. 21, the starting point of the diagnostic window is determined by identifying the start of a cardiac cycle and adding a fixed amount of time in some instances. As shown in the graphical representation 350 of FIG. 21, a start 352 of the cardiac cycle has been identified and a fixed time period 354 has been added to determine the starting point 356 of a diagnostic window. The fixed time period 354 is between about 150 ms and about 900 ms in some instances. In some instances, the fixed time period 354 is between about 300 ms and about 600 ms. In some particular embodiments, the fixed time period 354 is calculated as a percentage of the length 358 of the cardiac cycle of the patient. As shown in FIG. 21, an end 360 of the cardiac cycle has been identified such that the length 358 of the cardiac cycle extends between the start 352 and the end 360. The percentage of the length 358 of the cardiac cycle utilized for calculating the starting point 356 is between about 25% and about 95% in some instances. In some particular instances, the percentage of the length 358 of the cardiac cycle is between about 40% and about 75%. In yet other instances, no time is added to the start of the cardiac cycle, such that the start of the cardiac cycle 352 is the starting point 356.

Referring now to FIG. 22, the starting point of the diagnostic window is determined by identifying a ventricularization point in some instances. As shown in the graphical representation 362 of FIG. 22, a ventricularization point 364 of the cardiac cycle has been identified. In some instances, the ventricularization point 364 is identified based on the change in slope of the distal pressure reading. In the illustrated embodiment, the starting point 366 of the diagnostic window substantially coincides with the ventricularization point 364. In other instances, the starting point 366 is set to be a fixed amount of time before or after the ventricularization point. In that regard, the fixed time period is between about −250 ms and about 400 ms in some instances. In some instances, the fixed time period is between about −50 ms and about 100 ms.

Referring now to FIG. 23, shown therein is a graphical representation 370 illustrating a technique for identifying an ending point of a diagnostic window based on a starting point 372 of the diagnostic window. As shown, the diagnostic window has an ending point 374 that is spaced from the starting point 372 by a fixed amount of time 376. The fixed time period 376 is between about 1 ms and about 700 ms in some instances. In some instances, the fixed time period 376 is between about 200 ms and about 500 ms. In some particular embodiments, the fixed time period 376 is calculated as a percentage of the length of the cardiac cycle of the patient. The percentage of the length of the cardiac cycle utilized for calculating the time period 376 is between about 0% and about 70% in some instances. In some instances, the percentage of the length of the cardiac cycle is between about 25% and about 50%. In other instances, the diagnostic window is a specific point in the cardiac cycle such that time 376 is zero. In that regard, the techniques described for identifying the starting point and/or the ending point of a diagnostic window are suitable for identifying such a diagnostic point in the cardiac cycle for evaluating pressure differential. In some instances, a diagnostic window for a single cardiac cycle is comprised of a plurality of discrete diagnostic points along the single cardiac cycle.

Referring now to FIG. 24, shown therein is a graphical representation 380 illustrating a technique for identifying an ending point of a diagnostic window based on identifying the end of a cardiac cycle according to a proximal pressure measurement, which is an aortic pressure measurement in some instances, and subtracting a fixed amount of time. As shown, an end 382 of the cardiac cycle has been identified and a fixed time period 384 has been subtracted to determine the ending point 386 of a diagnostic window. The fixed time period 384 is between about 1 ms and about 600 ms in some instances. In some particular embodiments, the fixed time period 384 is calculated as a percentage of the length of the cardiac cycle of the patient. The percentage of the length of the cardiac cycle utilized for calculating the time period 384 is between about 0% and about 70% in some instances. In some instances, the percentage of the length of the cardiac cycle is between about 1% and about 25%. In yet other instances, no time is subtracted from the end of the cardiac cycle, such that the end of the cardiac cycle 382 is the ending point 386.

Referring now to FIGS. 25 and 26, shown therein are techniques for identifying an ending point of a diagnostic window based on a distal pressure measurement. Referring more specifically to FIG. 25, shown therein is a graphical representation 390 illustrating a technique for identifying an ending point of a diagnostic window based on identifying the end of a cardiac cycle according to a distal pressure measurement and subtracting a fixed amount of time. As shown, an end 392 of the cardiac cycle has been identified and a fixed time period 394 has been subtracted to determine the ending point 396 of a diagnostic window. The fixed time period 394 is between about 1 ms and about 600 ms. In some instances, the fixed time period 394 is between about 5 ms and about 100 ms. In some particular embodiments, the fixed time period 394 is calculated as a percentage of the length of the cardiac cycle of the patient. The percentage of the length of the cardiac cycle utilized for calculating the time period 394 is between about 0% and about 70%. In some instances, the percentage of the length of the cardiac cycle is between about 1% and about 25%. In yet other instances, no time is subtracted from the end of the cardiac cycle, such that the end of the cardiac cycle 392 is the ending point 396.

Referring to FIG. 26, shown therein is a graphical representation 400 illustrating a technique for identifying an ending of a diagnostic window based on identifying the ventricularization point of a distal pressure measurement. As shown, a ventricularization point 402 of the cardiac cycle has been identified. In some instances, the ventricularization point 402 is identified based on the change in slope of the distal pressure reading. In the illustrated embodiment, an ending point 404 of the diagnostic window substantially coincides with the ventricularization point 402. In other instances, the ending point 404 is set to be a fixed amount of time before or after the ventricularization point. In that regard, the fixed time period is between about −200 ms and about 450 ms. In some instances, the fixed time period is between about −50 ms and about 100 ms.

Referring now to FIGS. 27 and 28, shown therein are graphical representations of exemplary diagnostic windows relative to proximal and distal pressure measurements. In that regard, FIG. 27 illustrates a diagnostic window that begins shortly after ventricularization, while FIG. 28 illustrates a diagnostic window that begins before ventricularization.

Referring more specifically to FIG. 27, graphical representation 410 shows a diagnostic window 412 that includes a starting point 414 and an ending point 416. In some instances, the starting point 414 is selected using one or more of the techniques described above for identifying a starting point of a diagnostic window. Similarly, in some instances, the ending point 416 is selected using one or more of the techniques described above for identifying an ending point of a diagnostic window. As shown, the diagnostic window 412 begins after the ventricularization point of the distal pressure reading 304 and ends before the end of the cardiac cycle.

Referring now to FIG. 28, graphical representation 420 shows a diagnostic window 422 that includes a starting point 424 and an ending point 426. In some instances, the starting point 424 is selected using one or more of the techniques described above for identifying a starting point of a diagnostic window. Similarly, in some instances, the ending point 426 is selected using one or more of the techniques described above for identifying an ending point of a diagnostic window. As shown, the diagnostic window 422 begins before the ventricularization point of the distal pressure reading 304 and ends before the end of the cardiac cycle such that the ventricularization point is included within the diagnostic window 422.

Referring now to FIG. 29, shown therein is graphical representation of an ECG signal 440 annotated with exemplary diagnostic windows according embodiments of the present disclosure. Generally, at least one identifiable feature of the ECG signal 440 (including without limitation, the start of a P-wave, the peak of a P-wave, the end of a P-wave, a PR interval, a PR segment, the beginning of a QRS complex, the start of an R-wave, the peak of an R-wave, the end of an R-wave, the end of a QRS complex (J-point), an ST segment, the start of a T-wave, the peak of a T-wave, and the end of a T-wave) is utilized to select that starting point and/or ending point of the diagnostic window. For example, in some instances, a diagnostic window 442 is identified using the decline of the T-wave as the starting point and the start of the R-wave as the ending point. In some instances, the starting point and/or ending point of the diagnostic window is determined by adding a fixed amount of time to an identifiable feature of the ECG signal 440. In that regard, the fixed amount time is a percentage of the cardiac cycle in some instances.

Referring now to FIG. 30, shown therein is a graphical representation 450 of a proximal pressure 452 and a distal pressure 454 over a series of cardiac cycles of a patient. In that regard, a diagnostic window 456 has been identified that includes a starting point 458 and an ending point 460 for a cardiac cycle 462. The diagnostic window 456 is defined by the starting point 458 and the ending point 460. In the illustrated embodiment, the starting point 458 is selected to be positioned at a fixed percentage of the total diastole time of the cardiac cycle 462 after a maximum decline in pressure. In some instances, the fixed percentage of the total diastole time added to the point of maximum pressure decline to determine the starting point 458 is between about 10% and about 60%, with some particular embodiments having a percentage between about 20% and about 30%, and with one particular embodiment having a percentage of about 25%. The ending point 560 is selected to be positioned at a fixed percentage of the total diastole time or diastolic window from the beginning of diastole for the cardiac cycle 462. In some instances, the fixed percentage of the total diastole time added to the beginning of diastole to determine the ending point 460 is between about 40% and about 90%, with some particular embodiments having a percentage between about 60% and about 80%, and with one particular embodiment having a percentage of about 70%. In other embodiments, the ending point 560 is selected to be positioned at a fixed percentage of the total diastole time or diastolic window from the end of diastole for the cardiac cycle 462. In some instances, the fixed percentage of the total diastole time subtracted from the end of diastole to determine the ending point 460 is between about 10% and about 60%, with some particular embodiments having a percentage between about 20% and about 40%, and with one particular embodiment having a percentage of about 30%. Accordingly, in the illustrated embodiment, both the starting point 458 and ending point 460 are selected based on a proportion of diastole of the cardiac cycle 462. As a result, diagnostic windows defined using such techniques for multiple cardiac cycles may vary from cardiac cycle to cardiac cycle because the length of diastole may vary from cardiac cycle to cardiac cycle. As shown in FIG. 30, a diagnostic window 466 has been identified that includes a starting point 468 and an ending point 470 for a cardiac cycle 472 that follows cardiac cycle 462. As a result, the diagnostic window 466 will be longer or shorter than the diagnostic window 456, in some instances, because of differences in the length of diastole between cardiac cycle 462 and cardiac cycle 472.

While examples of specific techniques for selecting a suitable diagnostic window have been described above, it is understood that these are exemplary and that other techniques may be utilized. In that regard, it is understood that the diagnostic window is determined using one or more techniques selected from: identifying a feature of a waveform or other data feature and selecting a starting point relative to the identified feature (e.g., before, after, or simultaneous with the feature); identifying a feature of a waveform or other data feature and selecting an ending point relative to the identified feature (e.g., before, after, or simultaneous with the feature); identifying a feature of a waveform or other data feature and selecting a starting point and an ending point relative to the identified feature; identifying a starting point and identifying an ending point based on the starting point; and identifying an ending point and identifying a starting point based on the ending point.

In some instances, the starting point and/or ending point of a maximum diagnostic window is identified (using one or more of the techniques described above, for example) and then a portion of that maximum diagnostic window is selected for use in evaluating the pressure differential across a stenosis. For example, in some embodiments the portion selected for use is a percentage of the maximum diagnostic window. In some particular embodiments, the portion is between about 5% and about 99% of the maximum diagnostic window. Further, in some instances, the portion selected for use is a centered portion of the maximum diagnostic window. For example, if the maximum diagnostic window was found to extend from 500 ms to 900 ms of a cardiac cycle and a centered portion comprising 50% of the maximum diagnostic window was to be utilized as the selected portion, then the selected portion would correspond with the time from 600 ms to 800 ms of the cardiac cycle. In other instances, the portion selected for use is an off-centered portion of the maximum diagnostic window. For example, if the maximum diagnostic window was found to extend from 500 ms to 900 ms of a cardiac cycle and an off-centered portion comprising 25% of the maximum diagnostic window equally spaced from a mid-point of the maximum window and an ending point of the maximum window was to be utilized as the selected portion, then the selected portion would correspond with the time from 700 ms to 800 ms of the cardiac cycle. In some instances the diagnostic window is selected for each cardiac cycle such that the location and/or size of the diagnostic window may vary from cycle to cycle. In that regard, due to variances in the parameter(s) utilized to select the beginning, end, and/or duration of the diagnostic window from cardiac cycle to cardiac cycle, there is a corresponding variance in the diagnostic window in some instances.

Referring now to FIGS. 31 and 32, illustrated are timelines associated with a multi-modality data collection procedure. Specifically, FIG. 31 shows a graph 500 that is similar to graph portion 200 in FIG. 8. In particular, graph 500 depicts resistance (pressure over velocity) within a vessel of a patient over a plurality of heartbeat cycles. Using techniques discussed in association with FIG. 8, diagnostic windows 502, 504, and 506 have been identified. The diagnostic windows 502, 504, and 506 all correspond to the same section of the patient's heartbeat cycles. In the illustrated embodiment, the identified sections correspond to the portion of the heartbeat cycle of the patient where the resistance is reduced without the use of a hyperemic agent or other stressing technique. Data collection during a multi-modality diagnostic and/or treatment procedure may be temporally synchronized using the diagnostic windows 502, 504, and 506. That is, the diagnostic windows 502, 504, and 506 define the periods of time in which instruments utilized in the procedure may collect data.

The example of FIG. 31 illustrates a procedure timeline 508 associated with a multi-modality data collection procedure in which FFR (pressure) data is collected, OCT data is collected, and angiogram data is collected. As shown, data associated with each modality is collected during each of the diagnostic windows 502, 504, and 506. That is, (1) the sensor configured to collect OCT data is energized (and therefore collecting data) during the portions of the patient's heartbeat cycle represented by diagnostic windows 502, 504, and 506, (2) the angiogram machine captures images (x-ray or otherwise) during the diagnostic windows 502, 504, and 506, and (3) FFR evaluations are calculated using pressure data collected during the diagnostic windows 502, 504, and 506. In the procedure timeline 508 of FIG. 31, data collection with respect to each modality occurs concurrently during the diagnostic windows. However, in other multi-modality procedures, data collection may not occur concurrently.

In that regard, FIG. 32 illustrates the same graph 500 with diagnostic windows 502, 504, and 506, but shows a different procedure timeline 510. The procedure timeline 510 is associated with a multi-modality data collection procedure in which FFR (pressure) data is collected, OCT data is collected, and angiogram data is collected. However, in the timeline 500, although each modality data collection occurs during the same diagnostic window, the diagnostic windows represent sections of different cardiac cycles. For example, the FFR evaluation is calculated with pressure data collected during the diagnostic window 502, the OCT data is collected during diagnostic window 504, and the angiogram data is collected during the diagnostic window 506. Because all three modality data types are collected during the same section of a patient's heartbeat cycle, the data may be accurately co-registered for improved diagnosis. In one instance, each of the data-collecting instruments may capture data at any point during a diagnostic window. For example, in one embodiment, an instantaneous OCT snapshot of a patient's vessel may occur at any point during the diagnostic window. However, in other embodiments data, collection during a diagnostic window may be limited to specific points.

In that regard, FIG. 33 illustrates a graph 514 and an associated procedure timeline 516. The graph 514 is similar to the graphical representation 450 in FIG. 30. In particular, graph 514 depicts the proximal pressure 452 and the distal pressure 454 over a series of cardiac cycles 462 and 472 of a patient. Using techniques discussed in association with FIG. 30, diagnostic windows 456 and 466 have been identified. The procedure timeline 516 is associated with a multi-modality data collection procedure in which IVUS data is collected and angiogram data is collected. As shown, data associated with each modality is collected during each of the diagnostic windows 456 and 466. In the illustrated embodiment of FIG. 33, a chronological midpoint is defined for each of the diagnostic windows 456 and 466. For example, diagnostic window 456 has a midpoint 518 and diagnostic window 466 has a midpoint 520. In one embodiment, the IVUS and angiogram data collection occurs at the midpoints 518 and 520 of the respective diagnostic windows 456 and 466. In other embodiments, different points within the diagnostic window may be identified as data collection triggers. For example, instead of a data collection point based on time, a collection point may be based on the pressure values within the diagnostic window. In one instance, a collection point may be the instance within the diagnostic window corresponding to the median pressure value within the diagnostic window.

It is understood that the diagnostic windows and multi-modality data collection procedures described in association with FIGS. 31-33 are simply examples and that diagnostic windows may be established in a variety of different manners—for example, by using one or more of the techniques described above in association with FIGS. 8-30—and any number different and/or additional types of medical data may be collected within such diagnostic windows.

With reference now to FIG. 34, illustrated is a simplified flow chart describing a method 540 for collecting multi-modality data from a patient. In certain embodiments, the method 540 may be implemented and/or performed by portions of the medical system 100 of FIG. 1 including the multi-modality processing system 101.

The method 540 begins at block 542 where heartbeat data is acquired from a patient. The heartbeat data may be in many different forms including pressure data, velocity (flow) data, and/or ECG data. At least one cardiac cycle is identified using the heartbeat data. In one instance, pressure and flow data may combined to find resistance with a patient's vessel during a plurality of cardiac cycles, as illustrated in FIGS. 5-8. Next, in block 544, a diagnostic window is identified within a cardiac cycle of the patient. In one instance, the diagnostic window represents a portion of the heartbeat cycle that has a naturally reduced and relatively constant resistance. In other instances, diagnostic window encompasses the portion the heartbeat cycle that is less than a fixed percentage of the maximum resistance of the heartbeat cycle. The diagnostic window, as discussed in association with FIGS. 8-30 above, may be identified based on characteristics and/or components of one or more of proximal pressure measurements, distal pressure measurements, proximal velocity measurements, distal velocity measurements, ECG waveforms, and/or other identifiable and/or measurable aspects of vessel performance. In that regard, various signal processing and/or computational techniques can be applied to the characteristics and/or components of one or more of proximal pressure measurements, distal pressure measurements, proximal velocity measurements, distal velocity measurements, ECG waveforms, and/or other identifiable and/or measurable aspects of vessel performance to identify a suitable diagnostic window.

After a diagnostic window is established, the method 540 continues to block 546 where data in a first medical modality is acquired from the patient during the diagnostic window. In one instance, the data collection may occur at any point within the diagnostic window. In other instances, the data collection occurs at a particular point within the diagnostic window, such as the midpoint. The medical data may be data in any number of different modalities including OCT data, angiogram data, FFR (pressure) data, IVUS data, VH data, FL-IVUS data, IVPA data, CFR data, CT data, ICE data, FLICE data, intravascular palpography data, transesophageal ultrasound data, or any other medical data known in the art.

In block 548, data in a second, different medical modality is acquired from the patient during the diagnostic window. In certain embodiments, the processing system 101 is configured to control multiple different instruments to collect data during the diagnostic window. For instance the processing system 101 may energize an IVUS sensor during the diagnostic window while concurrently commanding an angiography system to capture images of a patient during the diagnostic window. As described above, in some instances, the data in both the first and second modalities are collected during the same diagnostic window within the same cardiac cycle. However, in other instances, medical data corresponding to the first modality will be collected during the diagnostic window in a first cardiac cycle and medical data corresponding to the second modality will be collected during the diagnostic window in a subsequent cardiac cycle.

Finally, the method ends at block 550 where the data in the first and second modalities are co-registered. For example, an OCT image acquired within a diagnostic cycle may be integrated with an angiogram image also acquired during the same diagnostic cycle. The integrated image may be used for diagnostic purposes. In one embodiment, a user interface (UI) application executing on the processing system 101 (FIG. 1) may present a co-registration graphical UI that may present and/or combine processed image or signaling data from multiple modalities. For instance, a UI application may display a patient's cardiac cycle adjacent to IVUS imaging data or may display an IVUS image overlaid with borders that were previously drawn on an OCT image. Such co-registration UI applications may acquire data from two medical data streams simultaneously to facilitate a real time co-registration workflow.

It is understood that the method 540 for collecting multi-modality data from a patient is simply an example and a variety of different and/or additional steps may be performed in the context of method 540. For example within the block 544, additional specific actions may be taken to identify the starting point and ending point of the diagnostic window. Such actions are described in association with FIGS. 16-28. Additionally, some or all of the blocks of method 540 may be combined and/or performed concurrently. For example, the data collection in blocks 546 and 548 may be performed concurrently. As another example, the heartbeat data collected in block 542 may be considered to be the data in a first medical modality in block 546.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A method of collecting multi-modality medical data from a patient, comprising:
acquiring heartbeat data from the patient using a heart-monitoring device, the heartbeat data identifying a first cardiac cycle of the patient;
selecting a diagnostic window within the first cardiac cycle of the patient, wherein the diagnostic window encompasses only a portion of the first cardiac cycle of the patient;
acquiring first medical data from the patient during the diagnostic window using a first medical device, the first medical data being associated with a first medical modality;
acquiring second medical data from the patient during the diagnostic window using a second medical device, the second medical data being associated with a second medical modality different than the first medical modality; and
co-registering the first medical data with the second medical data.

2. The method of claim 1, wherein the acquiring heartbeat data includes:
introducing at least one instrument into a vessel of the patient; and
obtaining from the at least one instrument pressure measurements within the vessel, the pressure measurements collectively defining a waveform of the cardiac cycle of the patient.

3. The method of claim 2, wherein the at least one instrument introduced into the vessel of the patient is one of a pressure-sensing catheter and a pressure-sensing guidewire.

4. The method of claim 2, wherein selecting the diagnostic window is based on one or more characteristics of the pressure measurements.

5. The method of claim 2, further including obtaining flow velocity measurements of a fluid flowing through the vessel from the at least one instrument.

6. The method of claim 5, wherein selecting the diagnostic window is at least partially based on one or more characteristics of the flow velocity measurements.

7. The method of claim 5, wherein selecting the diagnostic window is based on a combination of the pressure and the flow velocity measurements.

8. The method of claim 1, wherein the acquiring heartbeat data includes:
attaching an electrocardiograph (ECG) device to the patient; and
obtaining from the electrocardiograph (ECG) device a waveform of the cardiac cycle of the patient.

9. The method of claim 8, wherein selecting the diagnostic window includes defining the diagnostic window to encompass a portion of the waveform extending from a decline of a T-wave to a start of an R-wave.

10. The method of claim 1,
wherein the heartbeat data further identifies a second cardiac cycle of the patient;
wherein the diagnostic window encompasses a portion of the second cardiac cycle that is similar to the portion of the first cardiac cycle; and
wherein the acquiring second medical data from the patient is performed during the second cardiac cycle.

11. The method of claim 1, wherein the acquiring first medical data and the acquiring second medical data are performed concurrently.

12. The method of claim 1, wherein the first and second medical modalities are each a different one of intravascular ultrasound (IVUS) imaging, intravascular photoacoustic (IVPA) imaging, optical coherence tomography (OCT), forward looking IVUS (FL-IVUS), fractional flow reserve (FFR), coronary flow reserve (CFR), and angiography.

13. A method of collecting multi-modality medical data from a patient, comprising:
introducing at least one instrument into a vessel of the patient;
obtaining from the at least one instrument pressure measurements within the vessel for at least one cardiac cycle of the patient;
selecting a diagnostic window within the at least one cardiac cycle of the patient, wherein the diagnostic window encompasses only a portion of the at least one cardiac cycle of the patient;
acquiring first medical data from the patient during the diagnostic window using a first medical device, the first medical data being associated with a first medical modality;
acquiring second medical data from the patient during the diagnostic window using a second medical device, the second medical data being associated with a second medical modality different than the first medical modality; and
co-registering the first medical data with the second medical data.

14. The method of claim 13,
wherein the at least one cardiac cycle includes a first cardiac cycle and a second cardiac cycle;
wherein the diagnostic window encompasses similar portions of the first and second cardiac cycles;
wherein acquiring first medical data from the patient is performed during the first cardiac cycle; and
wherein the acquiring second medical data from the patient is performed during the second cardiac cycle.

15. The method of claim 13, wherein the acquiring first medical data and the acquiring second medical data are performed concurrently.

16. The method of claim 13, wherein the first and second medical modalities are each a different one of intravascular ultrasound (IVUS) imaging, intravascular photoacoustic (IVPA) imaging, optical coherence tomography (OCT), forward looking IVUS (FL-IVUS), fractional flow reserve (FFR), coronary flow reserve (CFR), and angiography.

17. The method of claim 13, wherein the at least one instrument introduced into the vessel of the patient is one of a pressure-sensing catheter and a pressure-sensing guidewire.

18. The method of claim 13, wherein selecting the diagnostic window is based on one or more characteristics of the pressure measurements.

19. The method of claim 13, further including obtaining flow velocity measurements of a fluid flowing through the vessel from the at least one instrument.

20. The method of claim 19, wherein selecting the diagnostic window is at least partially based on one or more characteristics of the flow velocity measurements.

21. The method of claim 19, wherein selecting the diagnostic window is based on a combination of the pressure and the flow velocity measurements.

22. A multi-modality medical data collection system, comprising:
a heart-monitoring device;
a first medical device configured to acquire first medical data associated with a first medical modality;
a second medical device configured to acquire second medical data associated with a second medical modality different than the first medical modality; and
a multi-modality processing system in communication with the heart-monitoring device,
the first medical device, and the second medical device, the processing system being configured to:
acquire heartbeat data from a patient using the heart-monitoring device, the heartbeat data identifying a first cardiac cycle of the patient;
select a diagnostic window within the first cardiac cycle of the patient, wherein the diagnostic window encompasses only a portion of the first cardiac cycle of the patient;
control the first medical device to acquire the first medical data from the patient during the diagnostic window; and
control the second medical device to acquire the second medical data from the patient during the diagnostic window.

23. The multi-modality medical data collection system of claim 22, wherein the multi-modality processing system is configured to acquire the first medical data and acquire the second medical data concurrently.

24. The multi-modality medical data collection system of claim 22, wherein the first and second medical modalities are each a different one of intravascular ultrasound (IVUS) imaging, intravascular photoacoustic (IVPA) imaging, optical coherence tomography (OCT), forward looking IVUS (FL-IVUS), fractional flow reserve (FFR), coronary flow reserve (CFR), and angiography.

25. The multi-modality medical data collection system of claim 22,
wherein the heart-monitoring device is a pressure-sensing instrument introduced into a vessel of the patient; and
wherein the multi-modality processing system is configured to obtain from the pressure-sensing instrument pressure measurements within the vessel, the pressure measurements collectively defining a waveform of the cardiac cycle of the patient.

26. The multi-modality medical data collection system of claim 25, wherein the pressure-sensing instrument is one of a pressure-sensing catheter and a pressure-sensing guidewire.

27. The multi-modality medical data collection system of claim 25, wherein the multi-modality processing system is configured to select the diagnostic window based on one or more characteristics of the pressure measurements.

28. The multi-modality medical data collection system of claim 22, wherein the heart-monitoring device is an electrocardiograph (ECG) device coupled to the patient; and wherein the multi-modality processing system is configured to obtain from the electrocardiograph (ECG) device a waveform of the cardiac cycle of the patient.

29. The multi-modality medical data collection system of claim 28, wherein the multi-modality processing system is configured to select the diagnostic window to encompass a portion of the waveform extending from a decline of a T-wave to a start of an R-wave.

30. The multi-modality medical data collection system of claim 22, wherein the multi-modality processing system is configured to co-register the first medical data with the second medical data.
